(19) United States
(12) Patent Application Publication
(10) Pub. No.: US 2007/0072938 A1
(43) Pub. Date: Mar. 29, 2007

(54) DRONABINOL TREATMENT OF DELAYED CHEMOTHERAPY-INDUCED NAUSEA AND VOMITING

(76) Inventor: Kevin Rose, Marietta, GA (US)

Correspondence Address: MAYER, BROWN, ROWE & MAW LLP, P.O. BOX 2828, CHICAGO, IL, 60690-2828, US

(21) Appl. No.: 11/383,387

(22) Filed: May 15, 2006

Related U.S. Application Data
(60) Provisional application No. 60/680,519, filed on May 13, 2005.
(60) Provisional application No. 60/694,675, filed on Jun. 29, 2005.
(60) Provisional application No. 60/703,420, filed on Jul. 29, 2005.

(51) Int. Cl. A61K 31/353

(52) U.S. Cl. 514/454

(57) ABSTRACT

In various embodiments, the present invention provides pharmaceutical compositions comprising delta-9-tetrahydrocannabinol and methods of administering such compositions prior to the administration of chemotherapy to prevent or reduce the development of delayed chemotherapy-induced nausea and vomiting.

This application claims priority to U.S. Provisional Application Ser. Nos. 60/680,519 filed May 13, 2005, 60/694,675 filed Jun. 29, 2005, and 60/703,420 filed Jul. 29, 2005, the entire contents of which are hereby incorporated by reference herein.

### FIELD OF THE INVENTION

The present invention relates to the use of pharmaceutical compositions comprising delta-9-tetrahydrocannabinol (“delta-9-THC” or “THC”) as a treatment for delayed chemotherapy-induced nausea and vomiting.

### BACKGROUND OF THE INVENTION

The diagnosis of cancer and the need for chemotherapy is a life-shattering event for most patients. Further, a major stress for patients diagnosed with cancer is the chemotherapy itself, including the delayed chemotherapy-induced nausea and vomiting (“CINV”).

CINV, defined as nausea and vomiting occurring more than 24 hours after chemotherapy and lasting for up to one week, is common, with at least 50% of patients experiencing delayed CINV following moderately emetogenic chemotherapy. Currently, CINV is treated with antiemetic agents. The primary goal of therapy with antiemetic agents is total response or prevention of CINV. Achieving a total response or prevention of CINV is important as the impaired quality of life (“QoL”) imparted by CINV can affect treatment outcomes when patients refuse chemotherapy because of severe adverse events (“AEs”).

In the brain the endogenous neurotransmitters dopamine and serotonin (“5-HT3”) are released in response to emetic stimuli and mediate nausea and vomiting. Standard antiemetic therapy with the 5-HT3 receptor antagonist, ondansetron, has been shown to be effective for relieving delayed CINV. However, many patients do not respond to ondansetron. Thus, there is a need for alternate agents that can provide relief alone or as part of a combination therapy regimen for CINV.

### SUMMARY OF THE INVENTION

In one embodiment, the present invention provides pharmaceutical compositions comprising delta-9-THC and to methods of administering such compositions to a patient in need of delta-9-THC therapy.

In another embodiment, the present invention provides pharmaceutical compositions comprising delta-9-THC and methods of administering such compositions prior to the administration of chemotherapy to prevent or to reduce the development of delayed CINV.

In yet another embodiment, the present invention provides pharmaceutical compositions comprising delta-9-THC and methods of administering such compositions prior to and after the administration of chemotherapy to prevent or to reduce the development of delayed CINV.

In still another embodiment, the present invention provides pharmaceutical compositions comprising delta-9-THC and ondansetron as well as methods of administering such compositions prior to and/or after the administration of chemotherapy to prevent or reduce the development of delayed CINV.

### BRIEF DESCRIPTION OF THE DRAWINGS

FIG. 1 is a flow chart that depicts the disposition of the patients throughout the clinical trial.

FIG. 2 is a bar graph that depicts the total response of the patients during active treatment.

FIG. 3 is a bar graph that depicts the absence of nausea during active treatment.

FIG. 4 is a bar graph that depicts the mean nausea intensity during active treatment.

FIG. 5 is a bar graph that depicts the mean episodes of vomiting/retching during active treatment.

FIG. 6 is a bar graph that depicts the total response observed from all treatment groups between Days 2 and 5.

FIG. 7 is a bar graph that depicts the absence of nausea observed from all treatment groups between Days 2 and 5.

FIG. 8 is a table that depicts the primary and secondary efficacy results observed from all treatment groups between Days 2 and 5.

FIG. 9 is a table that depicts the exploratory efficacy results observed from all treatment groups on Day 1.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

It is to be understood that any ranges, ratios, and ranges of ratios that can be formed by any of the numbers or data present herein represent further embodiments of the present invention. This includes ranges that can be formed that do or do not include a finite upper and/or lower boundary. Accordingly, the skilled person will appreciate that such ratios, ranges and values are unambiguously derivable from the data presented herein.

As used herein, the term “prevent” shall have its plain and ordinary meaning to one skilled in the art of pharmaceutical or medical sciences. Moreover, “prevent” shall mean to stop or hinder a chemotherapy side effect, such as nausea or vomiting, from occurring.

As used herein, the term “reduce” shall have its plain and ordinary meaning to one skilled in the art of pharmaceutical or medical sciences. In addition, “reduce” shall mean to diminish or decrease the number of occurrences, the duration, or the intensity, of a chemotherapy side effect, such as nausea or vomiting.

As used herein, the terms “treat” and “treating” shall have their plain and ordinary meaning to one skilled in the art of pharmaceutical or medical sciences. Further, “treat” and “treating” shall mean to prevent or reduce CINV.

As used herein, the term “nausea” shall have its plain and ordinary meaning to one skilled in the art of pharmaceutical or medical sciences. Moreover, “nausea” shall mean an unpleasant feeling in the abdomen or stomach usually associated with an aversion to food.

As used herein, the terms “vomit” or “vomiting” shall have their plain and ordinary meaning to one skilled in the art of pharmaceutical or medical sciences. In addition, “vomit” or “vomiting” shall mean the forcible or violent ejection of the stomach contents through the mouth, usually as coordinated, involuntary spasms of the respiratory and abdominal muscles.

As used herein, the term “retching” shall have its plain and ordinary meaning to one skilled in the art of pharmaceutical or medical sciences. “Retching” shall also mean the actual attempt to vomit, consisting of brief spasmodic contractions of the diaphragm, thoracic muscles, and abdominal muscles. Finally, “retching” shall incorporate “dry heaves.”

As used herein, the terms “delta-9-THC” or “THC” are understood to refer to both natural and synthetic delta-9-tetrahydrocannabinol (e.g., dronabinol), and includes all salts, isomers, enantiomers, esters, prodrugs and derivatives of delta-9-THC.

Natural cannabinoid compounds can be obtained from several sources, and are frequently obtained from Cannabis Sativa. Natural cannabinoids can be used as a therapeutic agent for the treatment of a variety of diseases. The primary active cannabinoid in cannabis, delta-9-THC, has received much attention for its psychoactive properties, but this compound also displays analgesic, anti-spasmodic, anti-convulsant, anti-tremor, anti-psychotic, anti-inflammatory, anti-emetic, and appetite-stimulant properties.

The endogenous cannabinoid system is an important pathway involved in the emetic response. Cannabinoids have been shown to prevent chemotherapy-induced emesis by acting at central CB1 receptors by preventing the proemetic effects of endogenous compounds such as dopamine and serotonin.

A synthetic version of delta-9-THC, dronabinol, has been developed for medicinal purposes and has been marketed in the U.S. and elsewhere as an oral formulation sold under the trade name, MARINOL®. MARINOL® has been approved for use in the treatment of nausea and vomiting following cancer chemotherapy in the United States since 1985. Effective doses of MARINOL® for use in the treatment of nausea and vomiting following cancer chemotherapy range from about 2.5 mg/day to about 40 mg/day.

THC and other cannabinoids bind to receptors in the endogenous cannabinoids system, a unique biological pathway involved in regulating nausea, vomiting, appetite, and other physiologic processes. Concentrations of these receptors exist in many brain regions, including the cerebral cortex, hypothalamus, cerebellum, and brainstem, where the vomiting center (located in the nucleus tractus solitarius of the medulla oblongata) is found.

In one embodiment, the present invention provides for the administration of a pharmaceutically effective amount of dronabinol to a patient in need thereof, prior to the patient receiving a dose of chemotherapy.

In another embodiment, the present invention provides for the administration of a pharmaceutically effective amount of dronabinol to a patient in need thereof, prior to and following the patient receiving a dose of chemotherapy.

In yet another embodiment, the present invention provides for the administration of a pharmaceutically effective amount of dronabinol and ondansetron to a patient in need thereof, prior to and following the patient receiving a dose of chemotherapy.

In one embodiment, compositions of the present invention are in the form of an orally deliverable dosage unit. The terms “oral administration” or “orally deliverable” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

Compositions of the present invention can be formulated as solid, liquid or semi-solid dosage forms. In one embodiment, such compositions are in the form of discrete dose units or dosage units. The terms “dose,” “dose unit,” and/or “dosage unit” herein refer to a portion of a pharmaceutical composition that contains an amount of a therapeutic agent suitable for a single administration to provide a therapeutic effect. Such dosage units may be administered one to a small plurality (e.g., 1 to about 4) times per day, or as many times as needed to elicit a therapeutic response. A particular dosage form can be selected to accommodate any desired frequency of administration to achieve a specified daily dose. Typically one dose unit, or a small plurality (e.g., up to about 4) of dose units, provides a sufficient amount of the active drug to result in the desired response or effect.

Alternatively, compositions of the invention can also be formulated for rectal, topical, transdermal, or parenteral (e.g., subcutaneous, intramuscular, intravenous and intradermal or infusion) delivery. In one embodiment, compositions of the invention can be formulated as a patch, gel, lotion, ointment, cream, or spray.

In another embodiment, a single dosage unit, be it solid or liquid, comprises a therapeutically and/or prophylactically effective amount of dronabinol and/or ondansetron. The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

It will be understood that a therapeutically and/or prophylactically effective amount of a drug for a patient is dependent inter alia on the body weight of the patient. A “patient” herein to which a therapeutic agent or composition thereof can be administered includes a human subject of either sex and of any age, and also includes any nonhuman animal, particularly a domestic or companion animal, illustratively a cat, dog, or a horse.

In various embodiments, compositions of the invention are in the form of solid dosage forms or dosage units. Non-limiting examples of suitable solid dosage forms include tablets (e.g., suspension tablets, bite suspension tablets, rapid dispersion tablets, chewable tablets, effervescent tablets, bilayer tablets, etc.), caplets, capsules (e.g., a soft or a hard gelatin capsule), powder (e.g., a packaged powder, a dispensable powder, or an effervescent powder), lozenges, sachets, cachets, troches, pellets, granules, microgranules, encapsulated microgranules, powder aerosol formulations, or any other solid dosage form reasonably adapted for oral administration.

In another embodiment, compositions of the invention can be in the form of liquid dosage forms or units. Non-limiting examples of suitable liquid dosage forms include solutions, suspension, elixirs, syrups, liquid aerosol formulations, etc.

In yet another embodiment, compositions of the present invention can be in the form of a metered dose inhaler, such as the metered dose inhaler outlined in co-pending U.S. application Ser. No. 11/361,463, which is incorporated herein by reference. Specifically, the present invention can be in the form of a metered dose inhaler comprising about 0.5% delta-9-THC, about 10% ethanol (dehydrated alcohol), and about 89.5% Propellant BFA-134a (1,1,1,2 tetrafluroethane). In another embodiment, the present invention can be in the form of a metered dose inhaler comprising about 2.0% delta-9-THC, about 10% ethanol (dehydrated alcohol), and about 88.0% Propellant BFA-134a (1,1,1,2 tetrafluroethane).

In one embodiment, the dose of delta-9-THC received by a patient according to methods of the present invention may be, for example, about 1 to about 50 mg, about 2 mg to about 20 mg, or about 2 mg to about 10 mg per day. For example, a patient according to methods of the present invention may receive about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0 or 50.0 mg of delta-9-THC per day. The doses described herein may be administered once to a small plurality of times per day, for example about 1, 2, 3, 4, 5, or 6 times per day.

In another embodiment, the dose of ondansetron received by a patient according to methods of the present invention may be, for example, about 1 to about 50 mg, about 2 mg to about 20 mg, or about 2 mg to about 10 mg per day. For example, a patient according to methods of the present invention may receive about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0 or 50.0 mg of ondansetron per day. The doses described herein may be administered once to a small plurality of times per day, for example about 1, 2, 3, 4, 5, or 6 times per day.

In one embodiment of the present invention, a patient receives a pharmaceutically effective amount of delta-9-THC approximately 72 hours to approximately 1 hour prior to the patient receiving a dose of chemotherapy. In another embodiment, the patent receives a pharmaceutically effective amount of delta-9-THC approximately 48 hours to approximately 1 hour prior to the patient receiving a dose of chemotherapy. In yet another embodiment, the patient receives a pharmaceutically effective amount of delta-9-THC approximately 24 hours prior to the patient receiving a dose of chemotherapy.

In one embodiment of the present invention, a patient receives a pharmaceutically effective amount of delta-9-THC the day after receiving a dose of chemotherapy. In another embodiment, the patient receives a pharmaceutically effective amount of delta-9-THC every day for up to 3 days after receiving a dose of chemotherapy. In yet another embodiment, the patient receives a pharmaceutically effective amount of delta-9-THC every day for up to 5 days after receiving a dose of chemotherapy. In still another embodiment, the patient receives a pharmaceutically effective amount of delta-9-THC every day for up to 7 days after receiving a dose of chemotherapy. In another embodiment, the patient receives a pharmaceutically effective amount of delta-9-THC every day for up to 30 days after receiving a dose of chemotherapy.

Compositions of the invention optionally comprise one or more additional pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Illustrative excipients include antioxidants, surfactants, adhesives, agents to adjust the pH and osmolarity, preservatives, thickening agents, colorants, buffering agents, bacteriostats, stabilizers, and penetration enhancers. Generally speaking, a given excipient, if present, will be present in an amount of about 0.001% to about 95%, about 0.01% to about 80%, about 0.02% to about 25%, or about 0.3% to about 10%, by weight.

Illustrative antioxidants for use in the present invention include, but are not limited to, butylated hydroxytoluene, butylated hydroxyanisole, potassium metabisulfite, and the like. One or more antioxidants, if desired, are typically present in a composition of the invention in an amount of about 0.01% to about 2.5%, for example about 0.01%, about 0.05%, about 0.1%, about 0.5%, about 1%, about 1.5%, about 1.75%, about 2%, about 2.25%, or about 2.5%, by weight.

In various embodiments, compositions of the invention comprise a preservative. Suitable preservatives include, but are not limited to, benzalkonium chloride, methyl, ethyl, propyl or butylparaben, benzyl alcohol, phenylethyl alcohol, benzethonium, or combination thereof. Typically, the optional preservative is present in an amount of about 0.01% to about 0.5% or about 0.01% to about 2.5%, by weight.

In one embodiment, compositions of the invention optionally comprise a buffering agent. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein. One or more buffering agents, if desired, are present in compositions of the invention in an amount of about 0.01% to about 5% or about 0.01% to about 3%, by weight.

The foregoing excipients can have multiple roles as is known in the art. For example, some flavoring agents can serve as sweeteners as well as a flavoring agent. Therefore, classification of excipients above is not to be construed as limiting in any manner.

These and many other aspects of the invention will be fully apparent to one of ordinary skill in the art in view of the example set forth below. The example provided herein is illustrative and is not to be construed as limiting the invention in any manner.

### EXAMPLE

A randomized, double-blind, placebo-controlled, parallel-group study evaluating the antiemetic efficacy and tolerability of oral dronabinol alone, dronabinol in combination with ondansetron, ondansetron alone, and placebo in patients receiving moderate to high emetogenic chemotherapy was conducted. All patients received dexamethasone 20 mg and ondansetron 16 mg intravenously prechemotherapy. Patients receiving dronabinol, ondansetron, or dronabinol plus ondansetron also received dronabinol 2.5 mg before and after chemotherapy on Day 1 (combined active treatment group); Group Placebo did not receive dronabinol. On Day 2, placebo or fixed doses of 10 mg dronabinol, 16 mg ondansetron, or dronabinol plus ondansetron were administered. On Days 3-5, patients received placebo, flexible doses of 10-20 mg dronabinol, 8-16 mg ondansetron, or dronabinol plus ondansetron. Rescue antiemetics were permitted after using the maximum dose of medication. The primary efficacy variable was total response (“TR”) to study medication (TR=nausea intensity<5 mm on a 100-mm visual analog scale, no vomiting/retching, no rescue antiemetic). Secondary efficacy parameters included nausea status and intensity, as well as episodes of vomiting/retching. Active treatments were compared with each other and placebo on Days 2-5, and statistical significance was determined if P≦0.05 (unadjusted). Exploratory analyses were conducted post hoc to examine the effect of combined active treatment on Day 1 versus placebo. TR and presence/absence of nausea were evaluated using the Fisher's Exact Test. Tolerability was assessed by physical examination and adverse events.

64 patients were randomized and 61 analyzed for efficacy. The overall efficacy results for TR, nausea status and intensity, as well as episodes of vomiting/retching are shown in the Table One listed below. On Days 2-5, TR was comparable for Groups Dronabinol and Ondansetron. The percentage of patients without nausea was significantly greater in all treatment groups versus placebo. Nausea intensity was significantly reduced by all treatments versus placebo. There were no significant differences among active treatments. All treatments were well tolerated.

On Day 1, in the combined active treatment group (n=50), significant improvement versus placebo (n=13) was observed for TR (79% versus 40%; P=0.024), mean nausea intensity (8 mm versus 31 mm; P=0.029), and absence of nausea (79% versus 38%; P=0.013), respectively.

TABLE ONE
Overall Efficacy Results (Day 2-5)

| | | Group | Group | Group | |
|---|---|---|---|---|---|
| | | Dro- | Ondan- | Dronabinol/ | Group |
| | | nabinol | setron | Ordansetron | Placebo |
| Measure | Units | (n = 17) | (n = 14) | (n = 17) | (n = 13) |
| Median | mg | 20 | 16 | 17.5-20 | 0 |
| daily dose | | | | dronabinol | |
| | | | | 12-16 | |
| | | | | ondansetron | |
| Total | % | 54 | 58^{†} | 47 | 20 |
| response* | (frequency/n) | (7/13) | (7/12) | (7/15) | (2/10) |
| Absence | % | 71^{†} | 64^{†} | 53^{†} | 15 |
| of nausea* | (frequency/n) | (10/14) | (9/14) | (9/17) | (2/13) |
| Mean | mm | 10.1^{†} | 24.0^{†} | 14.3^{†} | 48.4 |
| nausea | (n) | (14) | (14) | (17) | (13) |
| intensity^{‡} | | | | | |
| Mean | episodes/day | 0.2 | 1.3 | 0.7 | 1.3 |
| vomiting/ | (n) | (13) | (12) | (15) | (10) |
| retching* | | | | | |

*Cochran-Mantel-Haenszel.
^{‡}Analysis of variance.
^{†}P ≦ 0.05 versus placebo.

The incidence of treatment-emergent adverse events (“AEs”) was similar among active treatment groups (71%-88%); AE rate in placebo-treated patients was 50%. Diarrhea and fatigue were the most common AEs (11%).

The antiemetic effect of dronabinol for delayed CINV was comparable with ondansetron. Results for dronabinol plus ondansetron were similar to either agent alone. Dronabinol was well tolerated.

Methods

Patient Inclusion Criteria

Written informed consent was required for patients entering the trial. Patients 18 years or older were required to have malignancy that did not involve the bone marrow and be undergoing chemotherapy including a moderately to highly emetogenic regimen, 12 oxaliplatin at doses, or the combination of doxorubicin (60 mg/m2) with cyclophosphamide (600 mg/m2) with taxanes for the treatment of breast cancer.

Patients could be receiving concomitant radiation therapy other than abdominal radiation, or be changing from prior chemotherapy to a new moderately or highly emetogenic agent alone or in combination with other agents. Women were eligible for enrollment if they had a negative pregnancy test at baseline (Day 1) and would not become pregnant during the trial. In addition, patients had to have an estimated life expectancy of at least 6 weeks following chemotherapy treatment. Patients could not have received antiemetic therapy in the 7 days before chemotherapy and were required to have an Eastern Cooperative Oncology Group (“ECOG”) performance status of 0 to 2 at the screening visit.

Patient Exclusion Criteria

Patients were excluded if they had a history of anticipatory nausea and/or vomiting. Patients with primary malignancy of the brain, spinal cord, or nervous system; metastases to these sites; or leukemias or lymphomas that involve the bone marrow were excluded. Patients were not eligible for enrollment if they had a history of brain surgery, moderate to severe brain trauma, or other neurological disorder likely to affect central nervous system (“CNS”) functioning. Marijuana use within 30 days of baseline and antiemetic agents, including diphenhydramine, within 7 days before baseline were not permitted.

Patients with conditions that might interfere with study participation were excluded, including those patients who had a history or current diagnosis of psychotic disorder, had evidence of substance abuse disorder, had taken opiates or benzodiazepines not at a stable dose for 2 weeks, or had unstable medical conditions.

Patient Disposition

The intent-to-treat (“ITT”) population consisted of patients randomized into the trial who took at least 1 capsule of study medication, had a baseline (Day 1) efficacy evaluation, and had at least 1 post baseline efficacy evaluation (of any type). All efficacy analyses were based on the ITT population.

Study Design

This was a randomized, double-blind, placebo-controlled, parallel-group, 5-day study to evaluate the antiemetic efficacy and safety of oral dronabinol (sole under the trade name, Marinol®) alone, and in combination with ondansetron (sole under the trade name, Zofran®), versus ondansetron alone in patients receiving moderately to highly emetogenic chemotherapy. The investigator obtained written approval from the Institutional Review Board, and the study was conducted in accordance with the Declaration of Helsinki. All patients who had a follow-up visit were considered to have completed the study, whether or not they took study medication.

Patients who entered the study were to undergo cancer chemotherapy with moderately to highly emetogenic agents on Day 1 when they were randomized into 1 of 4 treatment groups: dronabinol alone (Group Dronabinol), ondansetron alone (Group Ondansetron), combination therapy with dronabinol and ondansetron (Group Dronabinol/Ondansetron), or placebo (Group Placebo). Dosing during the study period is shown in Table One. All patients received a standard prechemotherapy regimen of dexamethasone (20 mg) and ondansetron (16 mg). Patients in the 3 active treatment groups also received 2.5 mg dronabinol prechemotherapy and postchemotherapy. Placebo patients received matching placebo prechemotherapy and postchemotherapy on Day 1.

A kit of open-label antiemetics (metoclopramide 10 mg, prochlorperazine 5 mg, and prochlorperazine 25-mg suppository) was provided to the study participants for use on Days 1 through 8 as rescue medications to treat intolerable nausea and vomiting and/or retching after using the maximum dose of study medication at any dosing interval. Patients recorded the use of rescue medication and returned the unused portion of the kit.

Each morning, patients recorded the number of vomiting and/or retching episodes in the previous day. In addition, the patient recorded the daily presence or absence of nausea and its duration.

Efficacy Assessments/Definitions

The primary efficacy measure was the incidence of total response to treatment following administration of moderately to highly emetogenic chemotherapeutic agents. Total response was defined as no vomiting and/or retching, intensity of nausea <5 mm on a 100-mm Visual Analog Scale (VAS scale 0-100 mm; 0 mm=no nausea; 100 mm=intractable nausea), and no use of rescue medication.

The secondary efficacy analysis included patients who took rescue medication. However, analyses were conducted only on data collected before the use of any rescue medication. The secondary efficacy assessments were complete response, presence or absence of nausea, episodes of vomiting and/or retching, duration of nausea and vomiting and/or retching, intensity of nausea measured by VAS, ECOG (wellness), and QoL. Complete response for vomiting/retching was defined as no vomiting/retching, intensity of nausea of ≦30 mm on the VAS, and no use of rescue therapy. The presence or absence of nausea, episodes of vomiting and/or retching, and duration of nausea and vomiting and/or retching were assessed from daily patient telephone diary entries completed each morning to report the previous day. The patient was prompted through the Interactive Voice Response System to use the standard VAS to assess nausea intensity. The ECOG (wellness) assessment was clinician administered at screening and again on Day 6, Day 7, or Day 8 (or early termination). The possible range of the ECOG assessment was 0 to 4, where 0 was normal activity with no limitations and 4 was very sick, rarely out of bed.

The McCorkle Symptom Distress Scale (QoL assessment) evaluation was conducted on Day 1 (postchemotherapy) and again on Day 6, Day 7, or Day 8 (or early termination). The McCorkle Symptom Distress Scale consisted of 13 items to be rated on a scale of 1 to 5. Lower values indicate less distress (higher QoL). The total score was the sum of the scores from all 13 questions. The possible range for the total score was 13 to 65.

Safety Analyses

To assess the safety of the active treatments, physical examination, 12-lead electrocardiograph with rhythm strip, clinical laboratory analysis, and vital sign measurements were conducted. AEs and concomitant medications were also assessed.

Statistical Analyses

Statistical tests for differences between treatment groups were performed using a 2-sided test with a 0.050 level of significance. Computations for all results were performed using SAS® Version 8.2 computer software package, unless otherwise specified. For efficacy data, baseline was defined as Day 1. End point was defined as the value obtained on Day 5. In the computation of end point, values from a premature discontinuation visit were used in a last observation carried forward analysis. If the value at the discontinuation visit was missing, the last available postbaseline observation was used.

For the primary efficacy parameter, a logistic regression model was used for the primary analysis and a Cochran-Mantel-Haenszel (“CMH”) test stratified by pooled center was performed as a supportive analysis. Continuous secondary efficacy parameters were analyzed for all pairwise comparisons using a 2-way analysis of variance (“ANOVA”) with treatment and pooled center as fixed factors. For data that was not normally distributed, data were ranked, and an ANOVA was performed on the ranked scores. Categorical secondary efficacy parameters were analyzed for all pairwise comparisons using the CMH test stratified by pooled center. No adjustment for multiple comparisons was performed for the secondary efficacy parameters. No interactions were examined for the secondary efficacy parameters. Compliance with study medication was defined as taking all doses of supplied medication and was measured throughout the trial.

This study was originally designed to include a total of 464 patients to detect a difference between dronabinol and ondansetron with 80% power; however, the anticipated number of patients was not achieved because of difficulty in recruitment. This difficulty stemmed from patient reluctance to potential randomization to placebo because of the distress associated with highly emetogenic chemotherapy and the current commercial availability of active treatments. Because of the reduced number of patients, statistical analysis was not performed for number of episodes of vomiting and/or retching, duration of vomiting and/or retching, and duration of nausea.

Post-hoc Analysis

Exploratory analyses were conducted post hoc to examine the effect of dronabinol on the day of chemotherapy (Day 1). Fifty-two patients received dronabinol 2.5 mg before and after chemotherapy; 14 patients received placebo pre- and post-chemotherapy. All patients in the 3 active treatment groups were combined and compared with those patients receiving placebo. Categorical parameters (total response, complete response, and presence/absence of nausea) were evaluated using the Fisher Exact Test. P values for VAS nausea intensity scores were computed based on Wilcoxon rank sum test.

Results

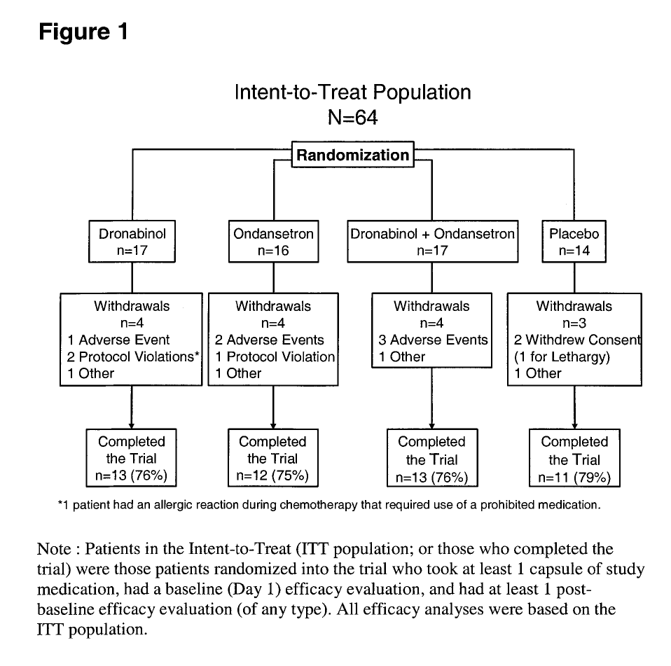

FIG. 1 shows the disposition of patients throughout the trial. Of the 64 patients who were randomized, 61 patients (95%) were included in the ITT population and 51 (80%) completed the trial. Of the 3 patients not included in the ITT population, 1 patient did not have chemotherapy, and 2 patients did not have a postbaseline efficacy evaluation. The primary cancer diagnosis of enrolled patients is shown below in Table Two. The most common diagnoses were breast cancer (26/64 patients, 41%) and non-small cell lung cancer (14/64 patients, 22%).

TABLE TWO
Primary Cancer Diagnosis

| Primary | | | | | |
|---|---|---|---|---|---|
| Cancer | | | Dronabinol/ | | All |
| Diagnosis, | Dronabinol | Ondansetron | Ondansetron | Placebo | Patients |
| n (%) | (n = 17) | (n = 16) | (n = 17) | (n = 14) | (n = 64) |
| Breast | 3 (18) | 5 (31) | 10 (59) | 8 (57) | 26 (41) |
| cancer | | | | | |
| Non-small | 5 (29) | 6 (38) | 1 (6) | 2 (14) | 14 (22) |
| cell lung | | | | | |
| cancer | | | | | |
| Colon, | 3 (18) | 0 | 1 (6) | 2 (14) | 6 (9) |
| rectal, or | | | | | |
| gastric | | | | | |
| cancer | | | | | |
| Lung | 1 (6) | 0 | 3 (18) | 1 (7) | 5 (8) |
| cancer | | | | | |
| (other) | | | | | |
| Ovarian | 1 (6) | 2 (13) | 0 | 0 | 3 (5) |
| cancer | | | | | |
| Prostate | 0 | 0 | 1 (6) | 1 (7) | 2 (3) |
| cancer | | | | | |
| Other | 1 (6) | 0 | 1 (6) | 0 | 2 (3) |
| small cell | | | | | |
| cancer | | | | | |
| Liver | 1 (6) | 0 | 0 | 0 | 1 (2) |
| cancer | | | | | |
| Kidney | 1 (6) | 0 | 0 | 0 | 1 (2) |
| cancer | | | | | |
| Pancreatic | 0 | 1 (6) | 0 | 0 | 1 (2) |
| cancer | | | | | |
| Hodgkin's | 1 (6) | 0 | 0 | 0 | 1 (2) |
| Non- | 0 | 1 (6) | 0 | 0 | 1 (2) |
| Hodgkin's | | | | | |
| Bladder | 0 | 1 (6) | 0 | 0 | 1 (2) |
| cancer | | | | | |

Patient demographics are presented below in Table Three. Most patients were aged 45 to 65 years. No statistically significant differences were noted between groups. The final median dosages of active medication on Days 3 through 5 were as follows: for Group Dronabinol, 20 mg/d; for Group Ondansetron, 16 mg/d; and for Group Dronabinol/Ondansetron, 17.5-20 mg/d of Dronabinol and 12-16 mg/d of Ondansetron.

TABLE THREE
Patient Demographics

| | Dronabinol | Ondansetron | Dronabinol/Ondansetron | Placebo | Overall |
|---|---|---|---|---|---|
| Parameter | (n = 17) | (n = 14) | (n = 17) | (n = 13) | (n = 61) |
| Age (y), | 61.6 ± 14.2 | 55.6 ± 16.1 | 56.8 ± 10.9 | 57.2 ± 8.6 | 57.9 ± 12.0 |
| mean ± SD | | | | | |
| Sex, n (%) | | | | | |
| Men | 9 (53) | 4 (29) | 6 (35) | 5 (38) | 24 (39) |
| Women | 8 (47) | 10 (71) | 11 (65) | 8 (62) | 37 (61) |
| Race, n (%) | | | | | |
| White | 13 (76) | 12 (86) | 13 (76) | 9 (69) | 47 (77) |
| Black | 1 (6) | 0 | 2 (12) | 3 (23) | 6 (10) |
| Hispanic | 1 (6) | 2 (14) | 2 (12) | 1 (8) | 6 (10) |
| Other | 2 (12) | 0 | 0 | 0 | 2 (3) |
| Prior | | | | | |
| marijuana | | | | | |
| use, n (%) | | | | | |
| Yes | 2 (12) | 1 (7) | 2 (12) | 1 (8) | 6 (10) |
| No | 15 (88) | 13 (93) | 15 (88) | 12 (92) | 55 (90) |
| Prior | | | | | |
| chemotherapy | | | | | |
| status, n | | | | | |
| (%) | | | | | |
| No | 15 (88) | 13 (93) | 14 (82) | 9 (69) | 51 (84) |
| Yes | 2 (12) | 1 (7) | 3 (18) | 4 (31) | 10 (16) |

ITT = intent to treat;
SD = standard deviation.
Weight not obtained in 1 dronabinol alone and 1 ondansetron alone patient Efficacy

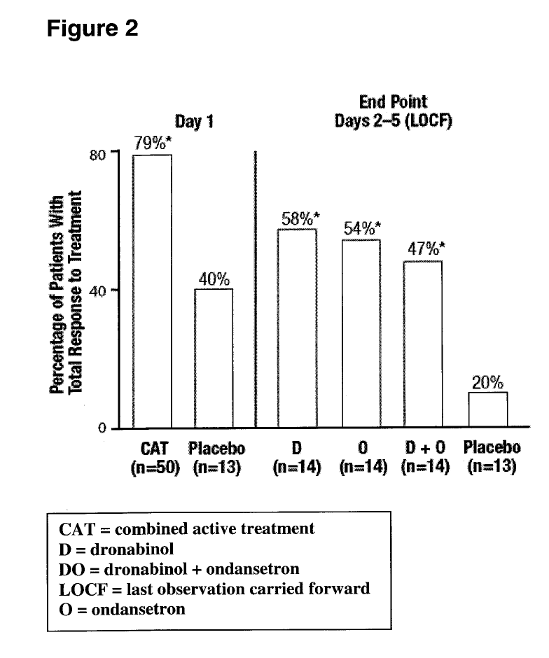

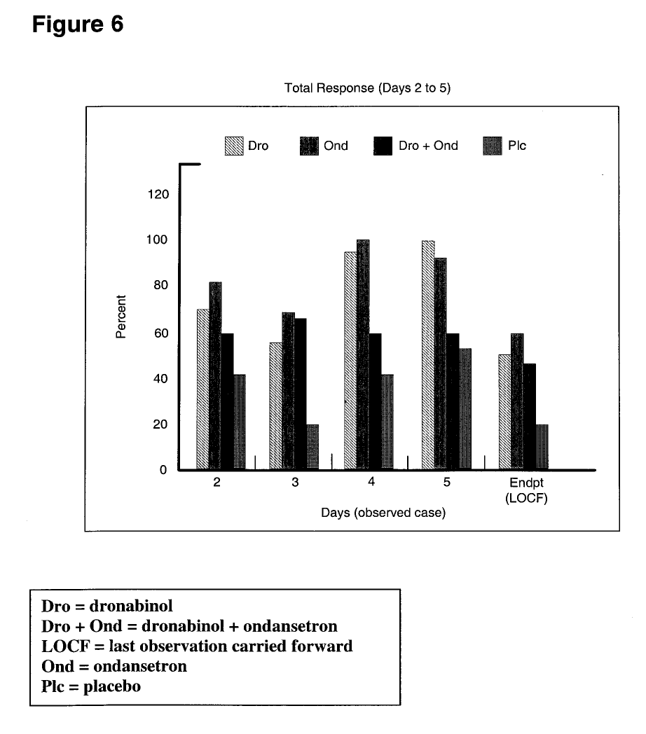

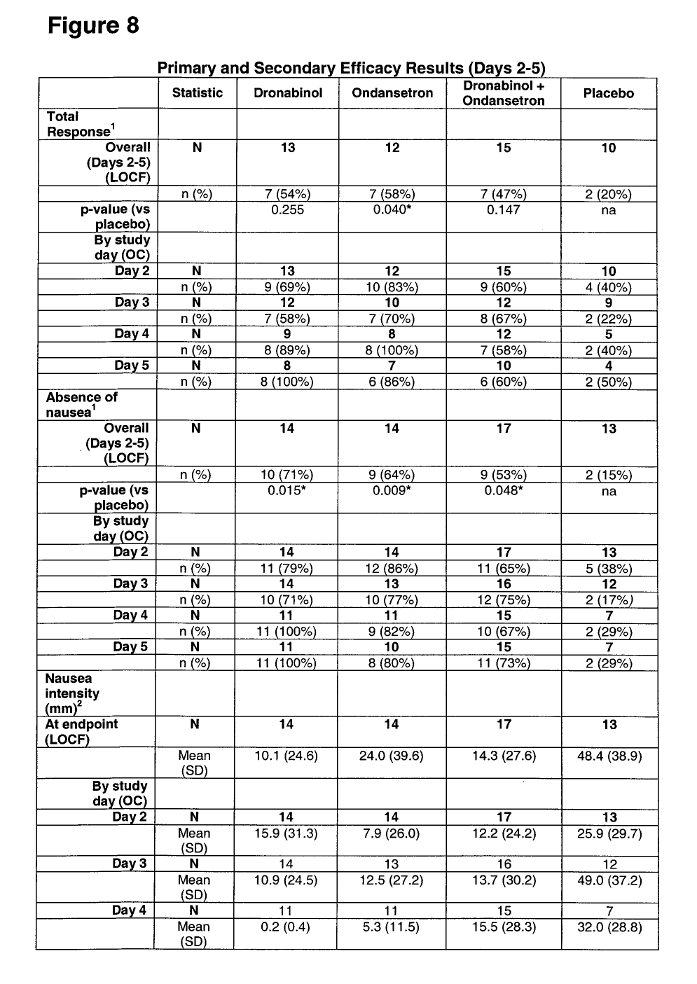

Total response (the primary efficacy variable) during the treatment phase is shown in FIG. 2. Group comparisons by day of the 3 treatment groups versus placebo on Days 2 through 5 were not statistically significant. Comparisons in total response rates at end point (Days 2-5 LOCF) showed that only patients in Group Ondansetron had a significantly greater total response than patients in Group Placebo (58% versus 20%; P=0.040). (See FIGS. 6, 8).

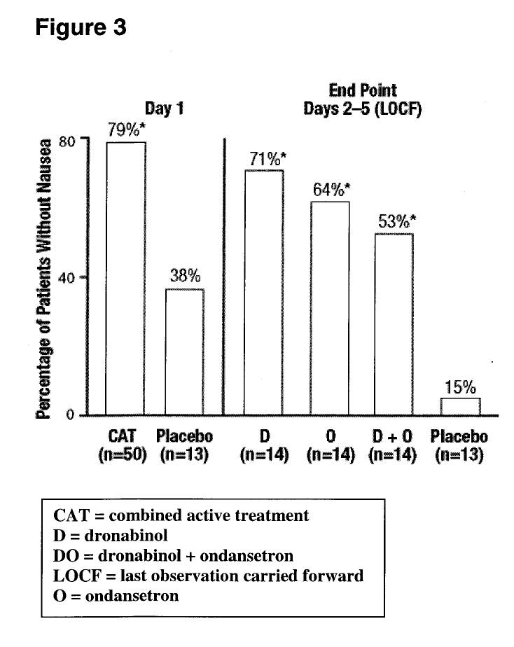

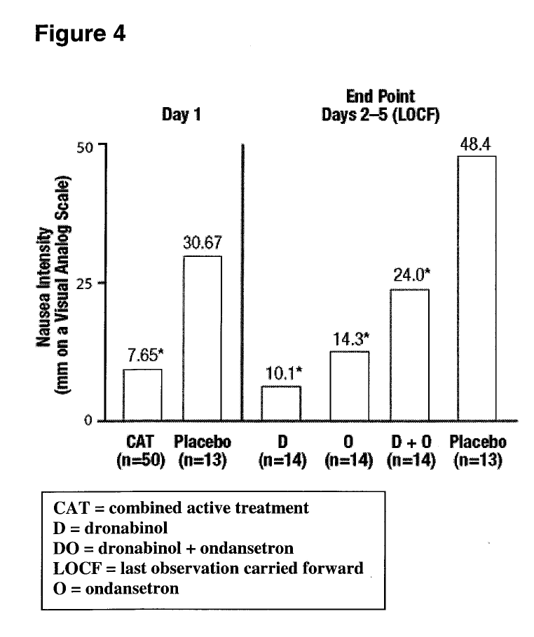

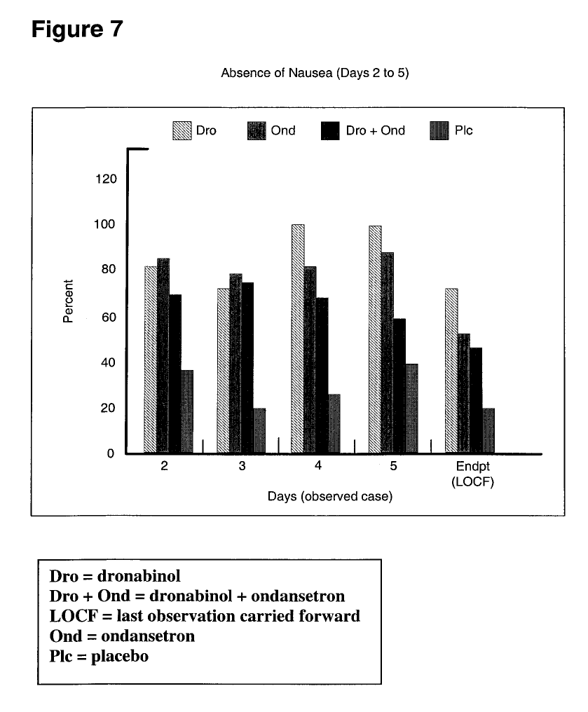

FIG. 3 shows that active treatment significantly increased the number of patients with no nausea at end point (Days 2-5 LOCF) in all treatment groups. (See FIG. 7). In addition, (FIG. 4) no significant difference in the intensity of nausea on the VAS was observed among groups at end point (Days 2-5 LOCF).

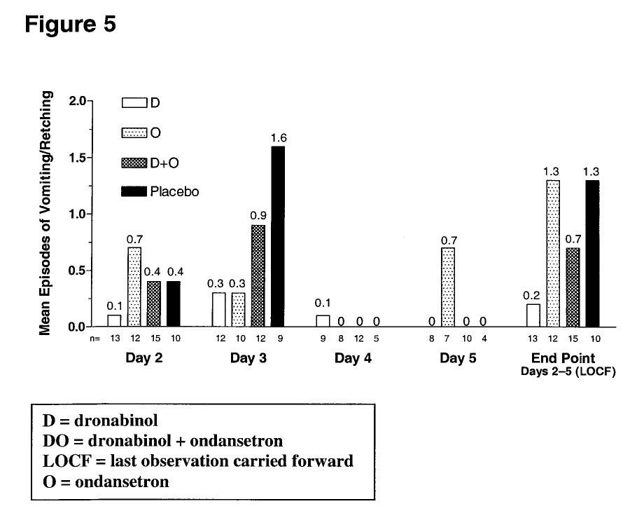

No significant difference was observed among groups for mean number of episodes of vomiting and/or retching (FIG. 5). Active treatment resulted in reducing the number of episodes of vomiting to 0 by Days 4 and 5 in some groups. Active treatment resulted in the reduction of the duration of vomiting/retching to 0 hours in all groups by Days 4 and 5 (Table Four below); duration of nausea (Table Five below) was comparable among groups.

TABLE FOUR
Duration of Vomiting/Retching
Observed Duration of Vomiting/Retching, hours (mean ± SD)

| | | | Dronabinol/ | |
|---|---|---|---|---|
| | Dronabinol | Ondansetron | Ondansetron | Placebo |
| | (n = 17) | (n = 14) | (n = 17) | (n = 13) |
| Day 2 | 0.00 ± 0.00 | 2.00 ± 6.93 | 0.04 ± 0.13 | 0.95 ± 3.00 |
| | (n = 12) | (n = 12) | (n = 14) | (n = 10) |
| Day 3 | 0.41 ± 1.36 | 0.06 ± 0.17 | 0.01 ± 0.03 | 1.57 ± 3.19 |
| | (n = 11) | (n = 9) | (n = 11) | (n = 7) |
| Day 4 | 0.00 ± 0.01 | 0.00 ± 0.00 | 0.00 ± 0.00 | 0.00 ± 0.00 |
| | (n = 9) | (n = 8) | (n = 12) | (n = 5) |
| Day 5 | 0.00 ± 0.00 | 0.02 ± 0.06 | 0.00 ± 0.00 | 0.00 ± 0.00 |
| | (n = 8) | (n = 7) | (n = 10) | (n = 4) |

SD = standard deviation.

TABLE FIVE
Duration of Nausea

| | | | Dronabinol/ | |
|---|---|---|---|---|
| | Dronabinol | Ondansetron | Ondansetron | Placebo |
| Parameter, n (%) | (n = 17) | (n = 14) | (n = 17) | (n = 13) |
| Day 2, n | 13 | 12 | 15 | 10 |
| No nausea | 9 (69) | 7 (58) | 9 (60) | 4 (40) |
| reported | | | | |
| <3 h | 1 (8) | 4 (33) | 3 (20) | 2 (20) |
| 3-6 h | 0 | 0 | 0 | 1 (10) |
| 6-8 h | 1 (8) | 0 | 0 | 2 (20) |
| 8-10 h | 0 | 0 | 1 (7) | 0 |
| >10 h | 2 (15) | 1 (8) | 2 (13) | 1 (10) |
| Day 3, n | 12 | 10 | 12 | 9 |
| No nausea | 7 (58) | 8 (80) | 6 (50) | 2 (22) |
| reported | | | | |
| <3 h | 2 (17) | 1 (10) | 2 (17) | 0 |
| 3-6 h | 0 | 0 | 0 | 2 (22) |
| 6-8 h | 2 (17) | 1 (10) | 2 (17) | 3 (33) |
| 8-10 h | 0 | 0 | 0 | 0 |
| >10 h | 1 (8) | 0 | 2 (17) | 2 (22) |
| Day 4, n | 9 | 8 | 12 | 5 |
| No nausea | 8 (89) | 7 (88) | 6 (50) | 2 (40) |
| reported | | | | |
| <3 h | 1 (11) | 1 (13) | 2 (17) | 2 (40) |
| 3-6 h | 0 | 0 | 2 (17) | 0 |
| 6-8 h | 0 | 0 | 2 (17) | 1 (20) |
| 8-10 h | 0 | 0 | 0 | 0 |
| >10 h | 0 | 0 | 0 | 0 |
| Day 5, n | 8 | 7 | 10 | 4 |
| No nausea | 7 (88) | 6 (86) | 6 (60) | 2 (50) |
| reported | | | | |
| <3 h | 1 (13) | 0 | 3 (30) | 2 (50) |
| 3-6 h | 0 | 1 (14) | 1 (10) | 0 |
| 6-8 h | 0 | 0 | 0 | 0 |
| 8-10 h | 0 | 0 | 0 | 0 |
| >10 h | 0 | 0 | 0 | 0 |

ITT = intent to treat;
IVRS = Interactive Voice Response System.

The complete responder rate is shown below in Table Six. Fifty eight percent of patients in Group Ondansetron and 60% of patients in Group Dronabinol/Ondansetron had significantly greater (P=0.04 and P=0.045, respectively) complete responder rates at end point on Days 2-5 (LOCF) versus Group Placebo (20%).

TABLE SIX
Complete Response

| | Treatment Group | | | | |
|---|---|---|---|---|---|
| Parameter, n (%) | CAT | D | O | DO | P |
| Day 1 | n = 42 | | | | n = 10 |
| Yes | 37 (88)) | | | | 7 (70) |
| No | 5 (12)) | | | | 3 (30) |
| LOCF End Point | | n = 13 | n = 12 | n = 15 | n = 10 |
| (Days 2-5) | | | | | |
| Yes | | 8 (62) | 7 (58)* | 9 (60)* | 2 (20) |
| No | | 5 (38) | 5 (42) | 6 (40) | 8 (80) |

*P < 0.05 vs P.
CAT = combined active treatment;
D = dronabinol;
DO = dronabinol plus ondansetron;
LOCF = last observation carried forward;
O = ondansetron;
P = placebo

According to the ECOG assessment (wellness), 41% to 69% of patients were rated as normal (not sick) in all treatment groups at baseline. Shifts in ECOG from 1 to 0 (improvement) occurred after treatment with dronabinol. However, a greater proportion of patients in the dronabinol group had a baseline value of 1, indicating that they were not very sick. Changes from baseline in ECOG were statistically significant in Group Dronabinol versus Group Placebo (P=0.036, in favor of Group Placebo) and Group Dronabinol versus Group Dronabinol/Ondansetron (P=0.028, in favor of Group Dronabinol/Ondansetron).

Improvement from baseline in McCorkle Symptom Distress Scale (QoL) was observed only in Group Dronabinol (mean change from baseline was −2.0±4.2). The only significant difference between groups for change from baseline was for Group Dronabinol versus Group Dronabinol/Ondansetron (mean change from baseline for Group Dronabinol/Ondansetron was +3.6±6.5; P=0.033, in favor of Group Dronabinol).

Other Analyses

Rescue antiemetics were used in all groups: dronabinol: 4/17 (24%), ondansetron: 5/16 (31%), dronabinol/ondansetron: 2/17 (12%), and placebo: 6/14 (43%). Rescue medicine use was low on Days 1 and 2 for all groups. There were no major differences between groups, except on Day 5, when half as many patients in Group Dronabinol (2/17, 12%) versus Group Ondansetron (4/16, 25%) required rescue medicine.

Compliance with study medication decreased over the course of treatment in all groups. A total of 29 subjects (45%) took all per-protocol doses of study medication over the full 5-day dosing period. At end point, (Days 1-5 LOCF), full compliance with study medication was higher in Group Dronabinol (59%) and Group Ondansetron (50%) than in Group Dronabinol/Ondansetron (35%) and Group Placebo (36%). No reasons for noncompliance were indicated.

Day 1

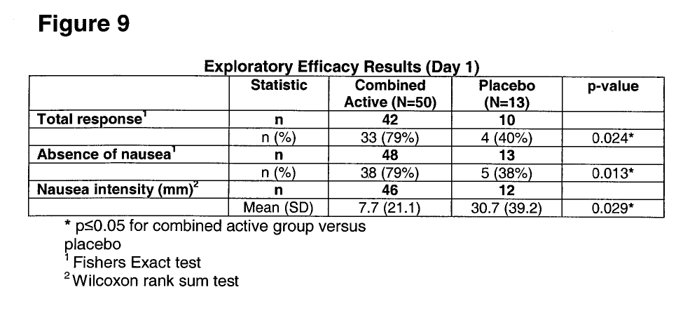

On Day 1, the results presented are for the combined active treatment group who took at least 1 dose of dronabinol (patients in Groups Dronabinol, Ondansetron, and Dronabinol/Ondansetron combined; n=50) versus the placebo group (n=13). As shown in FIG. 2, post-hoc analysis showed a significantly greater total response in the combined active treatment group compared with placebo (79% versus 40%; P=0.024). (See FIG. 9). No difference between groups was observed in the complete responder rate on Day 1 (Table Six). FIG. 3 shows that on Day 1, significantly more patients receiving active treatment had no nausea compared with those receiving placebo (79% versus 38%; P=0.013). In addition, (FIG. 4), the mean intensity of nausea scores on the VAS were significantly lower in the combined active treatment group (n=46) compared with placebo (n=12) on Day 1 (7.65% versus 30.67%; P=0.029).

Safety

As shown below in Table Seven, the incidence of treatment-emergent AEs was similar among active treatment groups (71%-88%); AE rate in Group Placebo was 50%. The highest rate of AEs was seen in Group Ondansetron. The highest rates of the CNS-related events of dizziness and fatigue were observed in Group Dronabinol/Ondansetron.

TABLE SEVEN
Summary of Treatment-Emergent Adverse Events (Safety Population)

| | Treatment Group | | | | |
|---|---|---|---|---|---|
| | D | O | DO | P | Overall |
| Parameter, n (%) | (n = 17) | (n = 16) | (n = 17) | (n = 14) | (n = 64) |
| Patients with at least 1 | 14 (82) | 14 (88) | 12 (71) | 7 (50) | 47 (73) |
| TEAE | | | | | |
| Patients with at least 1 | 2 (12) | 1 (6) | 1 (6) | 2 (14) | 6 (9) |
| SAE | | | | | |
| Patients with at least | 2 (12) | 1 (6) | 2 (12) | 3 (21) | 8 (13) |
| 1 severe TEAE | | | | | |
| Patients who | 1 (6) | 2 (13) | 3 (18) | 0 | 6 (9)* |
| permanently discontinued | | | | | |
| study medication | | | | | |
| because of a TEAE | | | | | |
| Patients with at least | 0 | 0 | 2 (12) | 0 | 2 (3) |
| 1 TEAE leading to | | | | | |
| dose reduction | | | | | |
| Adverse Events | | | | | |
| Occurring in 2 or | | | | | |
| More Patients | | | | | |
| Diarrhea | 4 (24) | 1 (6) | 1 (6) | 1 (7) | 7 (11) |
| Asthenia | 2 (12) | 1 (6) | 0 | 1 (7) | 4 (6) |
| Fatigue | 2 (12) | 1 (6) | 3 (18) | 1 (7) | 7 (11) |
| Chest pain | 1 (6) | 2 (13) | 0 | 0 | 3 (5) |
| Constipation | 1 (6) | 2 (13) | 1 (6) | 0 | 4 (6) |
| Dizziness | 1 (6) | 1 (6) | 4 (24) | 0 | 6 (9) |
| Headache | 0 | 3 (19) | 2 (12) | 0 | 5 (8) |
| Hyperglycemia | 0 | 2 (13) | 0 | 0 | 2 (3) |
| Insomnia | 0 | 2 (13) | 0 | 0 | 2 (3) |

D = dronabinol;
DO = dronabinol + ondansetron;
O = ondansetron;
P = placebo;
SAE = serious adverse event;
TEAE = treatment-emergent adverse event.
*Includes 2 patients (10161 and 10130) with adverse events that led to discontinuation (1 each from Groups DO and O) whose original reason for discontinuation was stated as “Normal End of Study.”

Discussion

This study demonstrated that the efficacy of dronabinol alone was comparable with ondansetron for the treatment of delayed CINV. This finding is important because standard antiemetic therapy does not relieve symptoms for many patients, and alternative treatments are necessary.

Because emesis is mediated by neurotransmitters in the CNS, patients receiving therapy with cannabinoids might be expected to have sensorial CNS AEs consistent with those reported in other trials with THC compounds. In this study, the highest rate of the CNS-related events of dizziness and fatigue were in Group Dronabinol/Ondansetron. The incidence of CNS-related events in Group Dronabinol was low. The CNS-related AEs reported in these previous studies may have been dose related considering that the dose of THC used was 50% greater than in the present study (30-45 mg daily versus a median dose of 20 mg/d, respectively). Well-tolerated and effective treatment of CINV, particularly for those refractory to treatment with standard antiemetics, may lead to improved treatment outcomes through improved compliance with chemotherapy. In this trial, compliance was highest in Group Dronabinol, although no formal statistical analysis was performed.

On Day 1, significantly greater efficacy on total response, absence of nausea, and intensity of nausea was demonstrated in the combined active treatment group compared with placebo. The data suggest that the addition of dronabinol before and after chemotherapy may offer more benefit than the standard regimen alone given before chemotherapy. However, because this study was not specifically designed to evaluate the effects of combined therapy on acute CINV, further studies are needed to validate the Day 1 findings. Significant improvement for treatment response on Day 1 may be important for the overall assessment of efficacy with dronabinol because it is believed that prevention of delayed CINV may be improved through effective control of acute CINV.

Acute CINV can be more severe than delayed CINV; however, the delayed symptoms can lead to hospitalization for dehydration and/or metabolic disorders that can have a greater effect on the patient's QoL. In this study, it was found that QoL was most improved in patients receiving dronabinol compared with patients in the other treatment groups.

The power of the study to detect statistically significant treatment group differences was reduced because the study was terminated early due to slow enrollment; however, the results were clinically meaningful. Treatment with dronabinol resulted in the highest rate for absence of nausea (71%) compared with ondansetron therapy (64%), combination therapy (53%), and placebo (25%). The data suggest that the addition of dronabinol to the standard antiemetic regimen before and after chemotherapy may offer more benefit than the standard regimen of ondansetron.

Conclusions

Dronabinol therapy (median dose, 20 mg/d) reduced delayed CINV with similar efficacy to ondansetron therapy (median dose, 16 mg/d). Dronabinol, ondansetron, and combination therapy had similar efficacy for total response, duration of nausea, and duration of vomiting/retching. However, either agent alone was generally superior to combination therapy or placebo. Dronabinol was well tolerated and produced few CNS-related AEs. The addition of dronabinol 2.5 mg to the standard antiemetic regimen before and after chemotherapy may offer many patients more benefit than the standard regimen alone before chemotherapy.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed:

1. A method of treating delayed chemotherapy induced nausea and vomiting comprising administering to a patient in need thereof a pharmaceutically effective amount of dronabinol prior to the patient receiving a dose of chemotherapy.

2. The method of claim 1, wherein the dronabinol is administered between about 24 hours to about 48 hours prior to a dose of chemotherapy.

3. The method of claim 1, wherein the pharmaceutically effective amount of dronabinol is about 2.5 mg/day to about 40 mg/day.

4. The method of claim 1, wherein the dronabinol is also administered to the patient after a dose of chemotherapy.

5. The method of claim 4, wherein the dronabinol is administered to the patient at least once a day for up to about five days after a dose of chemotherapy.

6. The method of claim 5, wherein the pharmaceutically effective amount of dronabinol is varied between the third day and the fifth day after a dose of chemotherapy.

7. The method of claim 6, wherein the pharmaceutically effective amount of dronabinol is increased.

8. The method of claim 1, wherein the dronabinol is administered to the patient in a dosage form selected from the group consisting of a metered dose inhaler, a capsule, a tablet, or a nasal spray.

9. The method of claim 1, wherein the dronabinol is administered to the patient through a transdermal delivery system.

10. The method of claim 9, wherein the transdermal delivery system is a patch.

11. A method of treating delayed chemotherapy induced nausea and vomiting comprising administering to a patient in need thereof a pharmaceutically effective amount of dronabinol prior to and following the patient receiving a dose of chemotherapy.

12. The method of claim 11, wherein the dronabinol is administered between about 24 hours to about 48 hours prior to a dose of chemotherapy.

13. The method of claim 11, wherein the pharmaceutically effective amount of dronabinol is about 2.5 mg/day to about 40 mg/day.

14. The method of claim 11, wherein the dronabinol is administered to the patient at least once a day for up to about five days after a dose of chemotherapy.

15. The method of claim 14, wherein the pharmaceutically effective amount of dronabinol is varied between the third day and the fifth day after a dose of chemotherapy.

16. The method of claim 15, wherein the pharmaceutically effective amount of dronabinol is increased.

17. The method of claim 11, wherein the dronabinol is administered to the patient in a dosage form selected from the group consisting of a metered dose inhaler, a capsule, a tablet, or a nasal spray.

18. The method of claim 11, wherein the dronabinol is administered to the patient through a transdermal delivery system.

19. The method of claim 18, wherein the transdermal delivery system is a patch.

20. A method of treating delayed chemotherapy induced nausea and vomiting comprising administering to a patient in need thereof a pharmaceutically effective amount of dronabinol and ondansetron prior to and following the patient receiving a dose of chemotherapy.
